(19) United States
(12) Patent Application Publication
(10) Pub. No.: US 2025/0093346 A1
(43) Pub. Date: Mar. 20, 2025

(54) OPTICAL INTERFEROMETRIC SENSOR

(71) Applicant: BIALOOM LTD, Nicosia (CY)

(72) Inventors: Thanasis MANOLIS, Nicosia (CY); Dimitris TSIOKOS, Attiki (GR); George TSEKENIS, Athens (GR)

(21) Appl. No.: 18/728,366

(22) PCT Filed: Jan. 21, 2022
(86) PCT No.: PCT/IB2022/050504

§ 371 Date: Jul. 11, 2024

(51) Int. Cl. G01N 33/543; G01N 21/45

(52) U.S. Cl. CPC G01N 33/54373; G01N 21/45

(57) ABSTRACT

An optical interferometric sensor presents a first optical path defining a sensing arm and a second optical path defining a reference arm. The sensor includes with respect to the first and second optical path an optical splitter placed upstream and an optical combiner placed downstream. Along the sensing arm are placed a first waveguide comprising a substrate and a binding surface functionalized to bind to at least one marker of an analyte and a first optical element. Along the reference arm are placed a second waveguide including a substrate identical to the substrate of the first waveguide and a second optical element presenting the same optical response of the first optical element. The sensor further includes a single microfluidic channel running through the first and the second waveguide.

### TECHNICAL FIELD

The present invention pertains to the technical field of photonic integrated circuits that use silicon photonics technology and plasmonics to implement a refractive index sensor on a planar chip.

Specifically, the refractive index sensor is transformed into a biosensor by applying specific binding chemistry on the sensing (plasmonic) surface thus providing an integrated plasmo-photonic biosensor.

Such a sensor can be advantageously implemented for medical analysis of fluid from a patient (e.g. blood, saliva, urine) and/or for food diagnostics, environmental monitoring or any other possible biochemical detection and measurement.

### BACKGROUND ART

One of the main problems to be addressed in the technical field of sensors is that of providing accurate and precise measurements.

Consequently, there is a great need for innovative solutions able to improve the sensitivity and precision of sensors, especially those meant to be implemented as biosensor for the detection of specific biochemical components and/or marker.

Furthermore, the increase in performances is normally counterbalanced by a corresponding increase in cost and complexity of the device.

As such, to develop a satisfactorily marketable product it is necessary to find a novel way to address the issue of performances without causing an excessive increase in its cost and complexity.

A kind of sensor of particular interest is that of optical interferometric sensors, which are characterized by the presence of a sensing arm wherein an analyte to be analysed can be allocated and a reference arm that provides a baseline for the analysis.

An optical signal can be fed to the interferometric sensors and split according to various techniques into the sensing and the reference arm.

Then by evaluating the output signal of the sensing arm against the one from the reference arm it is possible to acquire information about the analyte based on how the optical signal interacted with it.

Exploiting recent advances in nano-fabrication, plasmonic and photonic waveguide devices can be implemented into interferometric sensors, especially to be used as biosensors, to exploit phase dependence of the optical field at the waveguide to changes in refractive index of analyte under test.

However also this kind of devices is affected by the above-mentioned drawbacks and still requires major innovation to reduce complexity and cost while maintaining, if not improving, its performances.

### DISCLOSURE OF THE INVENTION

In this context, the technical purpose which forms the basis of this invention is to provide an optical interferometric sensor which overcomes at least some of the above-mentioned drawbacks of the prior art.

In particular, the aim of the invention is to provide an optical interferometric sensor with high Free Spectral Range that combines a simple design, small footprint and capable of optimal performances in any circumstance.

The technical purpose indicated and the aims specified are substantially achieved by a planarly integrated optical interferometric sensor comprising the technical features described in one or more of the appended claims.

The invention describes an optical interferometric sensor integrated on a chip, in particular of the Mach-Zender type, presenting a first optical path defining a sensing arm and a second optical path defining a reference arm.

In particular, the sensor presents a large Free Spectral Range which ranges from few tens of nanometres to several hundreds of nanometres.

From a structural point of view, the sensor essentially comprises an optical splitter, an optical combiner, a single microfluidic channel, a first and a second waveguide coupled to respective phase shifters.

In particular, the waveguides may be plasmonic or photonic waveguides, however plasmonic waveguides offer better sensing properties in smaller waveguide lengths.

More in detail, the optical splitter is positioned upstream of the first and second optical path and is configured to split an incoming optical signal equally into the sensing arm and the reference arm.

The optical combiner is instead placed downstream of the first and second optical path and is configured to recombine said optical signal after receiving the components previously split in the two arms of the interferometer.

The first waveguide is placed along the first optical path and comprises a substrate and a binding surface functionalized to interact and bind at least one marker of a sample.

Correspondingly, the second waveguide is placed along the second optical path and comprises a substrate identical to the substrate of the first waveguide.

Each waveguide is coupled to the respective phase shifter so that the sensor comprises a first optical element placed along the first optical path, for example downstream to the first waveguide and a second optical element placed along the second optical path, for example downstream to the second waveguide.

At least one of the optical elements comprise a phase shifter configured to tune the phase of an optical signal travelling in the respective arm.

Preferably, both the optical elements comprise respective phase shifter of which one is configured to tune the phase of an optical signal travelling in the respective arm, while the other simply balances the optical losses between the arms and does not need to be electrically biased. However, both elements can be biased if required during calibration.

The single microfluidic channel runs through both the first waveguide and the second waveguide.

Advantageously, the described sensor provides for a simple structure able to guarantee at the same time optimal measurements result, insofar the balancing between the sensing arm and the reference arm improves in a significant way the sensitivity and the noise cancelling capability of the sensor itself.

### BRIEF DESCRIPTION OF DRAWINGS

Further features and advantages of this invention are more apparent in the detailed description below, with reference to a preferred, non-restricting, embodiment of an optical interferometric sensor as illustrated in the accompanying drawings, in which:

FIG. 1 shows a schematic diagram of the optical interferometric sensor;

FIG. 2 shows more in detail a possible embodiment for one of the components of the sensor.

### DETAILED DESCRIPTION OF PREFERRED EMBODIMENTS OF THE INVENTION

In the accompanying figures with the reference numeral 1 is indicated in general an optical interferometric sensor, which for simplicity will be referred to in the following description simply as sensor 1.

In general, the sensor 1 defines an interferometer, preferably of the Mach-Zehnder type, which presents a first optical path 2 that defines a sensing arm and second optical path 3 defining a reference arm.

In other word the sensor presents a first optical path 2 along which a analyte can be allocated and a second optical path 3 that provide a reference for the measurements that are performed on the sample.

More in detail, the sensor 1 is an interferometric sensor with a large Free Spectral Range—FSR—which ranges from few tens of nanometres to several hundreds of nanometres.

Furthermore, the sensing arm and the reference arm presents a different length allowing to obtain a resonance in the recombined optical signal.

In general, the smaller the path difference is between the sensing and the reference arm of the interferometer, the larger the FSR becomes, resulting in higher interferometer sensitivity.

In this kind of device, the higher FSR results in a correspondingly higher noise that can be advantageously cancelled thanks to the specific structure and combination of elements described in the following.

From a structural point of view, the sensor 1 comprises an optical splitter 4 placed upstream of both the first and second optical paths 2, 3 and configured to split an incoming optical signal equally into the sensing arm and the reference arm.

Consequently, the sensor 1 may comprises an input line, for example an optical fibre, which can be coupled to an optical source, for example a tuneable laser source to feed the optical signal to the sensor 1.

The optical source may be an external source connected to the sensor 1 via the input line or directly in free space or the sensor 1 itself may comprises an integrated optical source to generate the optical signal without the need of being connected to a further device.

In use, the sensor 1 receives the optical signal from the light source and the optical splitter equally split said signal into the sensing arm, where it will interact with the analyte to be analysed, and into the reference arm.

Downstream from the sensing arm and the reference arm the sensor 1 also comprises an optical combiner 5 configured to recombine said optical signal after its components have travelled through the respective arm.

The recombined optical signal can then be processed to determine the properties of interest of the sample.

To this end, the sensor 1 can also comprise and/or be connected to one or more optical sensors configured to receive the recombined optical signal and preferably to determine/calculate at least one parameter representative of the recombined optical signal that can then be processed to identify the relevant properties of the analyte.

Along both the sensing arm and the reference arm are installed further components of the sensor 1 that allow to perform the desired measurement.

In particular, along the sensing arm are placed a first waveguide 6 and a first optical element 7 while along the reference arm are placed a second waveguide 8 and a second optical element 9.

Specifically, the waveguides are or comprise respective plasmonic or photonic waveguides.

Furthermore, at least one between the first optical element 7 and the second optical element 9 comprises a phase shifter configured to tune the phase of the optical signal in either the sensing arm or the reference arm (depending on which optical element comprises the phase shifter).

The presence of the phase shifter improves the optical sensitivity of the sensor, insofar it allows for an optical resonance shift by exploiting the strong light-matter interaction of the waveguide itself in large Free Spectral Range interferometer.

According to a possible embodiment, both the first optical element 7 and the second optical element 9 comprise a respective phase shifter configured to tune the optical signal in the sensing arm and in the reference arm respectively.

The presence of a second active phase shifter may be beneficial especially during a setup/calibration step of the sensor 1 to cancel out imperfections or noise (i.e. heat diffusing from the first phase shifter).

Alternatively, only one between the first optical element 7 and the second optical element 9 comprises a phase shifter thus presenting a certain optical response, while the other is simply structured in such a way as to present the same optical response without necessarily comprising a phase shifter of its own (e.g. by comprising an element structurally similar or identical to a phase shifter but which is not biased/powered).

The first waveguide 6 is thus installed along the first optical path 2 and structurally comprises a substrate S and a binding surface B functionalized to interact and bind at least one marker of the analyte.

The substrate S specifically comprises a couple of oxide layer presenting a first refractive index and a silicon nitride strip sandwiched between the oxide layers and presenting a second refractive index wherein the second refractive index is preferably higher than the first refractive index.

The binding surface B comprises instead at least one kind of recognition elements, preferably a biorecognition elements (A), configured to interact with and bind to a respective marker of the analyte specifically and selectively.

In other words, the binding surface B comprises a plurality of elements specifically selected/designed to interact with and bind to one or more substances, the markers, which the analysis being carried out aims to detect in the sample.

As an example, the biorecognition elements (A) may comprise at least one of the following: one or more antibodies, DNA strands, aptamers.

If the marker is present it is captured by the binding surface B, specifically by the recognition elements, thus affecting the optical signal that passes through the sensing arm.

Said interaction could be then identified in terms of modification of at least one parameters of the optical signal allowing to determine the presence and the properties (e.g. the amount and/or the concentration) of the marker in the analyte.

According to a preferred embodiment of the present application, the binding B surface presents a tridimensional structure that increases the exposed surface that can interact with the sample and the optical evanescent field to detect the desired marker.

Specifically, the binding surface B may comprise a plurality of tridimensional structures departing from the substrate S and defining a surface apt to support the (bio)recognition elements configured to bind the marker.

According to a possible embodiment of the present application, the binding surface B comprises a first plurality of tridimensional structures T1 and a second plurality of tridimensional structure T2.

The first plurality of tridimensional structures T1 presents a first height while the second plurality of tridimensional structures T2 presents a respective second height which is higher than the first height.

As such, the binding surface B presents overall a layered structure wherein a lower layer includes the first plurality of tridimensional structures T1 and the lower portion of the second plurality of tridimensional structures T2, while an upper layer includes the upper portion of the second tridimensional structure T2 which extends above the lower layer.

In this particular context the recognition elements A (for example a plurality of antibodies) are coupled to the second plurality of tridimensional structure T2 at a height higher than the first height.

In other words, the recognition elements A are integrally included in the upper layer, while no recognition element A is present in the lower layer.

In this way, the first plurality of tridimensional structure T1 is configured to define an antifouling surface that prevents the adsorption of undesired elements on the binding surface.

In other words, the marker of interest present in the sample interacts with the recognition elements A coupled to the upper portion of the second plurality of tridimensional structures T2, while the first plurality of tridimensional structures T1 prevents other substances to stick to the binding surface and consequently avoid their interference with the optical signal passing through the sensing arm.

In particular, it is preferable for the surface density of the first plurality of tridimensional structures T1 to be higher than a corresponding surface density of the second plurality of tridimensional structures T2.

In this way it is possible to maximise the antifouling effect and to provide for an optimal active surface area increasing the interaction between the recognition elements A with the analyte and the optical evanescent field.

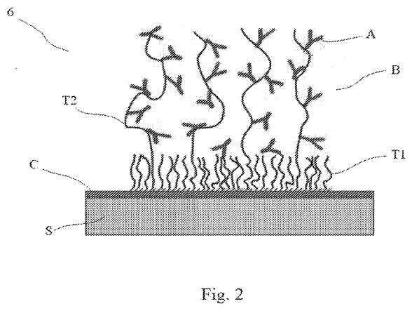

In general, the above discussed tridimensional structures may be constituted by a plurality of filament, as specifically shown in FIG. 2, or any analogous structure that provide for an increase in the active surface of the sensor 1, e.g. brush-like, tree-like or mesh-like structures.

Furthermore, the first waveguide 6 may also comprise a thin film coating C interposed between the substrate S and the binding surface B and configured to protect the substrate from oxidation.

To this end, the thin film coating C is preferably made of or comprises silicon nitride or an oxide.

The thin film coating C apart from protecting the substrate S against oxidation also allows more flexibility on the functionalization of the sensor, insofar normally only a limited number of alternative chemistries is available for the modification of the surfaces and the attachment of the recognition elements A, such as silanes whose deposition onto the surfaces is lengthy and requires numerous steps.

Addition of an extra layer on top of the substrate S increases the possible chemical functionalization routes that could be applied for the fabrication of the sensor 1 making it more versatile and facilitating its production.

For example, a thin film coating C made out of silicon nitride layer could allow for the (bio)functionalization of the sensor 1 with the use of hydrosilylation reactions, which is currently not possible.

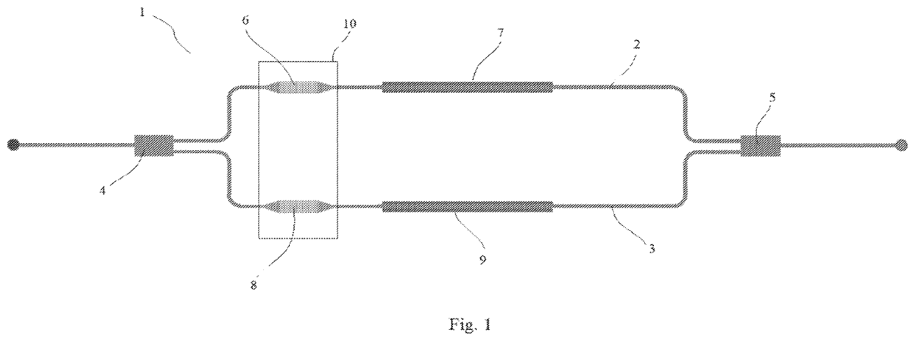

Along the first optical path 2, which defines the sensing arm of the sensor 1, is also present the first optical element 7, which as could be seen in FIG. 1 may be positioned downstream to the first waveguide 6.

The first optical element 7 may comprise a phase shifter that can be used to tune the phase of the optical signal in the sensing arm, specifically to tune the phase of the portion of the optical signal which is fed to the sensing arm by the optical splitter 4.

According to a possible embodiment, the first phase shifter 7 is defined by a couple of metal stripes directly deposited on top of the first waveguide 6 along the direction of propagation of the optical signal in the sensing arm.

The second waveguide 8 is instead installed along the second optical path 3 and structurally comprises a substrate S which is identical to the substrate of the first waveguide 6.

Furthermore, if the first waveguide 6 presents the above mentioned thin film coating C also the second waveguide 8 comprises a thin film coating C applied to its own substrate and identical both in structure and in function to the coating applied to the first waveguide 6.

According to a possible embodiment the second waveguide 8 can be an unfunctionalized waveguide that does not link with any specific marker in the analyte.

In this way the second waveguide 8 presents the same identical overall structure of the first waveguide 6 and differs from it only in that it does not capture any element of the analyte and thus the portion of the optical signal that is fed to the reference arm is only perturbated by the specific structure of the second waveguide 8 which mirrors that of the first waveguide 6.

To further improve the structural identity between the two distinct waveguide 6, 8, thus improving the noise cancelling capability of the sensor 1, the second waveguide 8 may also comprise a non-binding surface presenting the same optical response of the binding surface B comprised in the first waveguide 6 and configured not to bind with the marker.

In other words, the second waveguide 8 may comprise itself a structure superposed to the substrate S which mirrors the optical behaviour of the binding surface B, but which is specifically configured not to bind with the element comprised in the analyte and in particular not to bind with the marker.

For example, the non-binding surface comprises at least one kind of recognition elements A, which preferably are biorecognition elements comprising at least one kind of antibodies, for example a polyclonal mouse IgG.

Thus, the reference arm can also be biofunctionalized with a kind of recognition elements A that is not specific for the analyte of interest so that any eventual difference between the portion of the optical signal exiting the sensing arm and the one exiting the reference arm is solely linked to the interaction of the optical signal with the marker.

To this end the non-binding surface can present the same or similar structure described above for the binding surface S, i.e. it can present one or more tridimensional structures.

Along the reference arm there is also the second optical element 9, which as could be seen in FIG. 1 may be positioned downstream to the second waveguide 8 and in any case placed in such a way as to nearly mirror the structure and position of the first optical element 7 with reference to the first waveguide 6.

The second optical element 9 presents the same optical response of the first optical element 7 and consequently the portion of the optical signal interacting with it inside the reference arm behave in the same manner as the portion of the optical signal interacting with the first optical element 7 inside the sensing arm.

Specifically, if both the first optical element 7 and the second optical element 9 comprise respective phase shifters, they are both structurally and functionally identical and potentially configured to tune the phase of the optical signal, specifically of the portion of the optical signal, passing through their respective arm.

In this context, in a use configuration, one phase shifter (preferably the first optical element 7) remains preferably inactive.

The activation of a single phase shifter is sufficient to guarantee that the data of interest are located inside the window of observation of the sensor (data of interest is highly likely to shifts outside the window of observation in high FSR MZIs) while the other simply provide for a perfect structural balancing between the two arms thus allowing an optimal interference pattern and with higher extinction ration at the resonance without requiring to be powered.

In view of the above, and as shown in particular in FIG. 1, it can be seen that the sensor 1 presents perfectly balanced sensing and reference arm, wherein both arms comprise exactly the same/similar/corresponding elements and present the same geometry.

In known interferometric sensors the noise (specifically any thermal noise during the measurement or any other unwanted effect on the binding surface B) present on the sensing arm appears in the recombined optical signal thus limiting measurement resolution and limit of detection.

Specifically, the more the free spectral range of the interferometer is increased to increase sensitivity, the more the noise is also increased.

By applying exactly the same measurement conditions on the sensing and on the reference arm it is possible to cancel out the noise present in the sensing arm.

To further improve its sensing performances, the sensor 1 comprises a single microfluidic channel 10 running through both the first waveguide 6 and the second waveguide 8.

By flowing the same sample under test over the waveguide 6, 8 presents in each arm under the same experimental conditions, (temperature, sample flow, flow speed etc.) all noise will be cancelled out at the output of the sensor 1 even when the sensor 1 presents a high FSR, while modification of the optical signal will occur only for specific binding of targeted markers on the sensing arm.

According to a possible embodiment, the sensor 1 presents a plurality of optical path all enclosed between the optical splitter 4 and the optical combiner 5 and wherein one optical path defines the reference arm while all the other optical path defines respective sensing arm.

All sensing arm presents the same general structure and differs only in the specific functionalization of the binding structure B.

In particular, the binding structure B of each first waveguide 6 placed along each optical path is specifically functionalized to interact and bind with a specific and preferably distinct marker of the sample.

Also, in this situation the microfluidic channel 10 flows through all the optical path so that the measurement condition remains as stable as possible for each sensing arm.

In this way with a single analysis is possible to obtain information concerning a wide variety of marker in the analyte.

1. Optical interferometric sensor, in particular of the Mach-Zender type, presenting a first optical path defining a sensing arm and a second optical path defining a reference arm, said sensor comprising:
an optical splitter upstream of the first and second optical path configured to split an incoming optical signal equally into the sensing arm and the reference arm and an optical combiner placed downstream of the first and second optical path and configured to recombine said optical signal;
a first waveguide placed along the first optical path and comprising a substrate and a binding surface; said binding surface being functionalized to interact and bind at least one marker of a sample;
a first optical element placed along the first optical path;
a second waveguide placed along the second optical path and comprising a substrate identical to the substrate of the first waveguide;
a second optical element placed along the second optical path, and presenting the same optical response of the first optical element;
a single microfluidic channel running through the first waveguide and the second waveguide; and
at least one between the first optical element and the second optical element comprises a phase shifter configured to tune the phase of the optical signal in either the sensing arm or the reference arm.

2. Sensor according to claim 1, wherein each substrate comprises a couple of oxide layer presenting a first refractive index and a silicon nitride strip sandwiched between the oxide layers and presenting a second refractive index, said second refractive index being higher than the first refractive index.

3. Sensor according to claim 1, wherein the binding surface comprises at least one kind of recognition elements, preferably one kind of biorecognition elements, configured to bind specifically and selectively to a respective marker of said analyte.

4. Sensor according to claim 1, wherein the second waveguide is an unfunctionalized waveguide.

5. Sensor according to claim 1, wherein the second waveguide comprises a non-binding surface presenting the same optical response of the binding surface of the first waveguide and configured not to bind at least with the marker.

6. Sensor according to claim 6, wherein said non-binding surface comprises at least one kind of recognition elements selected so as not to bind with the marker, preferably said recognition elements comprising polyclonal mouse IgG antibodies.

7. Sensor according to claim 1, wherein the first waveguide and/or the second plasmonic guide comprise a thin film coating applied to the substrate and configured to protect the substrate from oxidation, preferably said thin film coating comprising silicon nitride or an oxide.

8. Sensor according to claim 1, wherein the first optical element and the second optical element comprise a respective phase shifter configured to tune the optical signal in the sensing arm and in the reference arm respectively.

9. Sensor according to claim 1, wherein the first optical element and/or the second optical element comprise a thermo-electric phase shifter.

10. Sensor according to claim 1, comprising:
an optical source upstream of the optical splitter and configured to generate the optical signal;
an optical sensor downstream of the optical combiner configured to receive a recombined optical signal

11. Sensor according to claim 1, wherein the binding surface comprises a plurality of tridimensional structures apt to carry the recognition elements.

12. Sensor according to claim 11, wherein the tridimensional structures comprise at least one between: a plurality of filament, tree-like structures, brush-like structures or mesh-like structures.

13. Sensor according to claim 11, wherein the binding surface comprises a first plurality of tridimensional structures presenting a first height and a second plurality of tridimensional structures presenting a second height higher than the first height, said recognition elements being coupled to the second plurality of tridimensional structures at a height higher than the first height.

14. Sensor according to claim 13, wherein the first plurality of tridimensional structures defines a antifouling surface.

15. Sensor according to claim 13, wherein a surface density of the first plurality of tridimensional structures is higher than a corresponding surface density of the second plurality of tridimensional structures.
